(19) United States
(12) Patent Application Publication
(10) Pub. No.: US 2006/0003030 A1
(43) Pub. Date: Jan. 5, 2006

(54) Essential oils for treating and/ or preventing allergic disease

(76) Inventors: Lin Chun-Ying, Taipei (TW); Hsu Chih-Chieh, Taipei (TW); Hsu Hui-Chun, Taipei (TW); Yang Wen-Jia, Taipei (TW); Tsai Ying-Chieh, Taipei (TW)

Correspondence Address: HARNESS, DICKEY & PIERCE, P.L.C., P.O. BOX 8910, RESTON, VA, 20195, US

(21) Appl. No.: 10/880,651

(22) Filed: Jul. 1, 2004

(51) Int. Cl. A61K 35/78

(52) U.S. Cl. 424/725; 424/769

(57) ABSTRACT

The present invention provides the essential oil and the essential oil composition of traditional Chinese herbal medicine to prevent and/or treat allergic disease. The essential oil and the essential oil composition can be formulated as a solution, a cream, a lotion, ointment, or a solid stick, and applied topically.

### FIELD OF THE INVENTION

The present invention relates to the novel use and compositions of herbal medicine for treating and/or preventing allergic disease.

### BACKGROUND OF THE INVENTION

Allergy refers to any condition of the body mounting an attack on a specific foreign substance. People can experience allergic reactions to foods, chemicals, plants, animals and a variety of airborne substances. The substance to which the person is allergic is called the allergen. Allergy is defined as a hypersensitivity or hyperimmunity caused by exposure to a particular antigen (allergen) resulting in a marked increase in reactivity to that antigen/allergen upon subsequent exposure. The symptoms of allergic diseases, such as allergic rhinitis (hay fever), allergic dermatitis, and allergic asthma, can be caused by a variety of atopic allergens, such as grasses, trees, weeds, animal dander, insects, molds, drugs, and chemicals. For example, allergic rhintis is a very common allergic disease worldwide and is a kind of type 1 hypersensitivity. Allergic rhintis refers to an inflammatory condition of nose cause by allergy and affects millions of people throughout the world. The typical symptoms of allergic rhinitis include sneezing, itchy nose, nasal congestion and rhinorrhea (“running” or “runny” nose), often accompanied by watery and itchy eyes, and postnasal drip. These allergic diseases are mediated by an antibody known as immunoglobulin E, or simply IgE. Anti-IgE medicines that reduce IgE levels are attractive treatments for allergy patients.

IgE is a member of the immunoglobulin family that mediates allergic responses such as asthma, food allergies, type 1 hypersensitivity, and the familiar sinus inflammation suffered on a widespread basis. IgE bonds to mast cells and basophiles. Upon combination of a specific allergen with IgE bound to mast cells or basophiles, the IgE may be cross-linked on the cell surface, resulting in the physiological effects of IgE-antigen interaction. This may result in the release of histamine, serotonin, heparin, a chemotactic factor for eosinoplylic leukocytes and/or the leukotrienes, C4, D4, and E4, which cause prolonged constriction of bronchial smooth muscle cells. These released substances are the mediators, which result in allergic symptoms.

Immunosuppressant compounds induce an inhibition of the immune response system and are customarily used in the treatment of allergic disease. Compounds which are known to exhibit immunosuppressant activity include the fungal metabolite Cyclosporin A and the macrolide antibiotic (a metabolite from Streptomyces tsukabaensis) termed FK506. Both of these agents have been used clinically and experimentally to suppress the immune system in transplantation and in the treatment of a number of diseases. As for allergic rhintis, some pharmaceuticals, such as antihistamines, decongestants, corticosteroids and mast cell stabilizers, are used for treating and preventing allergic rhinitis. However, they are not necessarily satisfactory, because they are not entirely effective against the nasal congestion of allergic rhinitis and have side effects such as sleepiness, malaise, and sedation due to inhibitory action on the central nervous system (Ju Lih-Ling, The Journal of Pharmacy, Volume 58, page 79-86, 1999). In contrast, NSAIDS are also very useful as anti-inflammatory drugs. In addition, NSAIDS and anti-inflammatory steroid agents have a cytokine-production suppressing effect and have also been used for autoimmune diseases.

Pharmaceutical agents for allergic diseases have been developed and used for the therapy. However, since such pharmaceutical agents have adverse effects, there has been a strong demand for anti-allergic agents derived from natural products, where long-term administration is possible, safety is high, and no adverse reaction takes place.

### SUMMARY OF THE INVENTION

One object of the invention is to provide a composition for use in the treatment and/or prevention of allergic disease, which comprises the essential oil of HOUXIANG HERBA, the essential oil of QIANGHUO HERBA and the essential oil of XINYI HERB, wherein HOUXIANG HERBA comprises a plant selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indicathe; QIANGHUO HERBA comprises a plant selected from the group consisting of Notopterygium forbesii and Notopterygium incisum; and XINYI HERB comprises a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri.

Another object of the invention is to provide a method of treating and/or preventing allergic disease, comprising administering an effective and sufficient amount of essential oil of HOUXIANG HERBA to a patient for treating and/or preventing allergic disease, wherein HOUXIANG HERBA comprises the plants selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indica. Preferably, the essential oil is obtained from Agastache rugosa.

A further object of the present invention is to provide a method of treating and/or preventing allergic disease, comprising administering an effective and sufficient amount of essential oil of QIANGHUO HERBA (RHIZOMA ET RADIX NOTOPTERYGH) to a patient for treating and/or preventing allergic disease, wherein QIANGHUO HERBA comprises the plants selected from the group consisting of Notopterygium forbesii and Notopterygium incisum.

### BRIEF DESCRIPTION OF THE DRAWING

FIG. 1 shows the average number of nasal rubbings for each test group.

FIG. 2 shows the average number of sneezes for each test group.

### DETAILED DESCRIPTION OF THE INVENTION

The present invention provides methods for the treatment of allergic disease by using plant essential oils and the pharmaceutical composition comprising the plant essential oils. According to the invention, the term “essential oils” refers to the highly scented droplets found in minute quantities in the flowers, stems, leaves, root and barks of aromatic plants. They are not true oils in the manner of lubricant vegetable oils, but highly fluid and exceptionally volatile. Essential oils are complex mixtures of different organic molecules—terpenes, alcohols, esters, aldehydes, ketones, and phenols. In view of the volatility and pleasant aroma, plant essential oils have advantageous benefits such as their reduction of subjects' complaints about uncomfortable administration and their ease of use.

Compositions of Herbal Essential Oils

One aspect of the invention is to provide a composition for use in the treatment and/or prevention of allergic disease, which comprises the essential oil of HOUXIANG HERBA and the essential oil of XINYI HERB, wherein HOUXIANG HERBA comprises a plant selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indicathe and XINYI HERB comprises a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri. Preferably, HOUXIANG HERBA is Agastache rugosa and XINYI HERBA is Magnolia biondii. According to one embodiment of the invention, the essential oil of HOUXIANG HERB in the composition ranges from 0.05% to 0.95% by volume, and the essential oil of XINYI HERB in the composition ranges from 0.05% to 0.95% by volume.

Another aspect of the invention is to provide a composition for use in the treatment and/or prevention of allergic disease, which comprises the essential oil of QIANGHUO HERBA and the essential oil of XINYI HERB, wherein QIANGHUO HERBA comprises a plant selected from the group consisting of Notopterygium forbesii and Notopterygium incisum and XINYI HERB comprises a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri. Preferably, QIANGHUO HERBA is Notopterygium forbesii and XINYI HERB is Magnolia biondii. According to one embodiment of the invention, the essential oil of QIANGHUO HERB in the composition ranges from 0.05% to 0.95% by volume, and the essential oil of XINYI HERB in the composition ranges from 0.05% to 0.95% by volume.

Another aspect of the invention is to provide a composition for use in the treatment and/or prevention of allergic disease, which comprises the essential oil of HOUXIANG HERBA, the essential oil of QIANGHUO HERB and the essential oil of XINYI HERB, wherein HOUXIANG HERB comprises a plant selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indicathe; QIANGHUO HERBA comprises a plant selected from the group consisting of Notopterygium forbesii and Notopterygium incisum; and XINYI HERB comprises a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri. Preferably, HOUXIANG HERBA is Agastache rugosa, QIANGHUO HERBA is Notopterygium forbesii and XINYI HERB is Magnolia biondii. According to one formulation of the invention, the composition comprises 0.5% to 99.0% by volume of the essential oil of HOUXIANG HERBA, 0.5% to 99.0% by volume of the essential oil of QIANGHUO HERBA, and 0.5% to 99.0% by volume of the essential oil of XINYI HERB. Preferably, the composition comprises 0.5% to 49.5% by volume of the essential oil of HOUXIANG HERBA, 0.5% to 49.5% by volume of the essential oil of QIANGHUO HERBA, and 50.0% to 99.0% by volume of the essential oil of XINYI HERB.

HOUXIANG HERBA is a known Chinese herbal medicine and used for treating gastrointestinal diseases such as abdominal pain, gastritis, enteritis, and gastrotympanities. According to the invention, HOUXIANG HERBA refers to a plant selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indica. It is known that the plants of HOUXIANG HERBA have similar pharmaceutical effects in Chinese medicine (Zhou De-Zhen, Master Thesis, Graduate School of Industry and Commerce, Chinese Culture University, 1970). Therefore, the plants, Agastache rugosa, Pogostemon cablin, Pogostemon formosanum, Microtoena delavayi, Microtoena insuavis, Microtoena patchouli, Teucrium quadrifarium, Epimeredi indica and Anisomeles indica, are mutually replaceable when used as pharmaceuticals.

XINYI HERB is a kind of traditional Chinese medicine and belongs to Magnoliaceae family. According to the invention, XINYI HERB refers to a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri. The herb is collected in early spring when the bud is not in blossom, dried in the sun, and cleared of stalks. It is known that the herb is often used for the treatment of chronic rhinitis, allergic rhinitis, and maxillary sinusitis. Kobayashi et al. indicated that magnosalin, a compound derived from Fols magnoliae, has inhibitory effects on the proliferation of synovial cells in rheumatoid archritis models (Kobayashi et al., Immunopharmacology 39(2): 139-147, 1998 May). The herb is traditionally taken orally. Furthermore, Wang (W. K. Wang et al., Journal of Shaanxi College of Tradition Chinese Medicine, 2000, Vol. 23., No. 2, pg. 40-42) has described Flos magnoliae as inhibiting allergy when administered orally.

QIANGHUO HERBA (RHIZOMA ET RADIX NOTOPTERYGH) is a traditional Chinese medicine and used in treating osteonecrosis, gout, arthritis, brain diseases, wounds, mixed neoplasms, digestive system neoplasms, and connective and soft tissue neoplasms (CN 99108294 and CN 98122088). According to the invention, QIANGHUO HERBA refers to a plant selected from the group consisting of Notopterygium forbesii and Notopterygium incisum. Notopterygium forbesii and Notopterygium incisum belong to Notopterygium family and have similar pharmaceutical effects.

According to the invention, the composition is further comprised of an essential oil, such as eucalyptus oil, lemon oil, menthol oil, almond oil, jojoba oil, rosehip oil, sesame oil, coconut oil, evening primrose oil, anise oil, cinnamon oil, lavender oil, bergamot oil, or avocado oil. Preferably, the other essential oil is selected from among one of eucalyptus oil, lemon oil, menthol oil, or bergamot oil. In addition, the composition also is further comprised of another agent for treating allergic rhinitis or chronic rhinitis known to persons skilled in the art, such as menthol oil, α-adrenergic decongestionants, (pseudoephedrine, phenylpropanolamine, oximethazoline, tramazoline), adrenergic bronchodilatators (epinephrine, isoproterenol, salbutamol, terbutaline, pirbuterol and metaproterenol), antihistamines (azelastine, levocabastine), corticosteroids (beclomethasone dipropionate, flunisolide), or the more preferable sodium chromoglycate and ipatropium bromide.

New Use of Essential Oils of HOUXIANG HERBA and QIANGHUO HERBA

Another aspect of the invention is to provide a method of treating and/or preventing allergic disease, comprising administering an effective and sufficient amount of essential oil of HOUXIANG HERBA to a patient for treating and/or preventing allergic disease, wherein HOUXIANG HERBA comprises the plants selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indica. Preferably, the essential oil is obtained from Agastache rugosa.

A further aspect of the invention is to provide a method of treating and/or preventing allergic disease, comprising administering an effective and sufficient amount of essential oil of QIANGHUO HERBA to a patient for treating and/or preventing allergic disease, wherein QIANGHUO HERBA comprises the plants selected from the group consisting of Notopterygium forbesii and Notopterygium incisum. Preferably, the essential oil is obtained from Notopterygium forbesii.

Preparation and Formulation of Herbal Essential Oils

According to the invention, the essential oils of the above-mentioned herbs are prepared by extraction methods known in the art. Preferably, the essential oil of the plants is extracted by water distillation.

According to the invention, the essential oil or the essential oil composition can be formulated as a solution, a cream, a lotion, an ointment, or a solid stick. The formulations can be directly applied to the topical skin of whole body, and the transdermal essential oil or the essential oil composition can infiltrate the skin to take effect. Especially when applying to the area between the nose and upper lip, the essential oil or the essential oil composition volatilizes and then can be administered to the nasal and respiratory mucus by inhalation.

For the solution formulation, the essential oil or the essential oil composition is optionally diluted with organic solvents that are non-toxic to humans, such as menthol, ethanol and isopropanol. The solution formulation can also be delivered by any one of a variety of inhalation devices. The term “inhalation device” refers to a device used to administer an essential oil/or other volatile oils as well known to persons skilled in the art, such as liquid nebulizers, sprayers, thermal vaporizers, burners, aromatherapy lamps, ultrasonic vaporizers, steamers, electrohydrodynamic aerosolizers, etc. In addition, the inhalation device must be practical, in the sense of being easy to use and durable. Preferably, the essential oil or the essential oil composition is delivered by using an aromatherapy lamp.

For the cream, ointment, or lotion formulation, the essential oil or the essential composition can be formulated in a suitable carrier. To prepare the essential oil or the essential composition in lotion, cream or ointment form, the essential oil or the essential composition is first dissolved in suitable carriers such as water, ethanol, propylene glycol, and/or another vehicle, and the solution thus obtained is mixed with a desired base to make lotion, cream or ointment.

For the stick formulation, the essential oil or the essential oil composition is combined with appropriate carriers to form a solid stick. The methods for preparing the stick are known in the art. Preferably, the carrier is menthol. The stick formulation can be directly applied to the topical skin of whole body and the transdermal essential oil or the essential oil composition can infiltrate the skin to take effect. Especially when applying to the area between the nose and upper lip, the essential oil or the essential oil composition volatilizes and then can be administered to the nasal and respiratory mucosa by inhalation.

According the invention, the essential oil or the essential oil composition can be administered by inhalation, percutaneous absorption or oral administration. Preferably, the essential oil or the essential oil composition is delivered by inhalation to achieve a reduced side effect and a fast uptake by the nasal and respiratory mucosa to relieve the symptoms of the allergic rhinitis or chronic rhinitis. The quantity of the essential oil or the essential oil composition administrated, and the duration of administration of a single dose all depend on the type of inhalation device employed and the form of the formulation, as those skilled in the art would recognize.

For inhalation the essential oil is administered by using a liquid nebulizer, a sprayer, thermal vaporizer, a burner, aromatherapy lamp, an ultrasonic vaporizer, a steamer, or an electrohydrodynamic aerosolizer. Normally, the amount of the essential oil or the essential oil composition ranges from about 10% to 100% by volume. Preferably, using an aromatherapy lamp, the formulations of the essential oil or the essential oil composition typically include the essential oil in a solution at an amount of about 10% to 15% by volume.

For percutaneous absorption the essential oil or the essential oil composition can be produced as an essential oil patch, mask, cream, or lotion. The essential oil can penetrate the skin and achieve a therapeutic effect.

For oral therapeutic administration, the essential oil may be diluted in water and/or potatory liquid.

According to the present invention the dose agent amount is administered to a patient ranges from about 0.01 ml/day to 0.05 ml/day. Preferably, the dose agent amount is administered to a patient at 0.02 ml/per day. Administration of the essential oil is contemplated to include chronic, acute, or intermittent regimens, and any mode where essential oil is feasible may be selected.

Utility

According to the invention, the essential oil or the essential oil composition can be used in the treatment and/or prevention allergic disease. When a mammal in exposed under an allergen, the immune system will be induced to produce the specific antibody IgE. The allergen binds the IgE to form an Ag-Ab (allergen-antibody) complex, so that macrophage and mast cell releases NO, histamine, platelet-activating factor (PAF) and other factors. According to the invention, the allergic diseases include allergic rhinitis, allergic pneumonia, allergic conjunctivitis, allergic dermatitis, allergic sinusitis, allergic asthma, allergic bronchitis, allergic arthritis, and allergic arthritis and food allergies. Preferably, the allergic disease is allergic rhinitis.

### EXAMPLE

### Example 1

### Inhibitory Effect of Essential Oils on NO Production by LPS-Activated Mouse Macrophage Cell Line RAW 246.7

A) Cell Culture

The mouse macrophage cell line RAW 264.7 was purchased from Bioresources Collection & Research Center, Taiwan and numbered CCRC 60001. The cells were added to Dulbecco's Modified Eagle Medium (DMEM, GibcoBRL) containing 10% (v/v) fetal bovine serum (FBS, GibcoBRL) and then incubated in a CO₂ incubator containing 5% CO₂ at 37° C. The cells grow to 90% density and are then washed with phosphate buffered saline (PBS). The cells were colleted and diluted with fresh DMEM to the appropriate concentration.

B) Determination of Production of Nitric Oxide

The mouse macrophage cells were transferred in a 96-well plate in a concentration of 10,000 cells/well. After incubating for 24 hours, the cells were activated with the medium containing 100 μg/ml Lipopolysaccharide (LPS, SIGMA) and 100·μg/ml Interferon-γ (INF-γ, GibcoBRL). Meanwhile, the essential oils of herbal medicine in different concentrations were added to the wells. After incubating for 24 hours, 50 μl of supernatants were respectively taken and put into a 96-well plate. 50 μl Griess reagent A (60 mM sulfanilamide solved in 3 N HCL) and 50 μl Griess reagent B (4 mM naphthylethylene diamine dihydrochloride) were respectively added to the cell supernatants and reacted at room temperature for 10 minutes. Then, the absorbance of the resulting cell supernatants was determined with a microplate reader at a wavelength of 500 mm. The percentage of nitric oxide can be calculated by the following formula:
% nitric oxide=(sample absorptivity/control absorptivity)×100%.

The results are shown in below Table 1.

TABLE 1

| | Concentration | | Nitric oxide |
|---|---|---|---|
| Samples | (μg/ml) | | (μM) |
| Blank | | | 3.5 ± 0.3 |
| LPS (Inducer) | | | 60 ± 0.5 |
| L-NMMA (positive control) | | | 25 ± 0.6 |
| Magnolia biondii | 0.1 | μg/ml | 40.8 ± 0.4 |
| Magnolia biondii | 1 | μg/ml | 37.8 ± 0.2 |
| Magnolia biondii | 10 | μg/ml | 33.1 ± 0.3 |
| Agastache rugosa. | 0.1 | μg/ml | 40.8 ± 0.5 |
| Agastache rugosa | 1 | μg/ml | 33.6 ± 0.4 |
| Agastache rugosa | 10 | μg/ml | 30.2 ± 0.3 |
| Notopterygium forbesii | 0.1 | μg/ml | 49.7 ± 0.2 |
| Notopterygium forbesii | 1 | μg/ml | 45.5 ± 0.3 |
| Notopterygium forbesii | 10 | μg/ml | 43.1 ± 0.2 |
| Magnolia biondii + Agastache | 0.1 | μg/ml | 35 ± 0.3 |
| rugosa (1:1 by volume) | | | |
| Magnolia biondii + Agastache | 1 | μg/ml | 30 ± 0.2 |
| rugosa (1:1 by volume) | | | |
| Magnolia biondii + Agastache | 10 | μg/ml | 27 ± 0.1 |
| rugosa (1:1 by volume) | | | |
| Magnolia biondii + Notopterygium | 0.1 | μg/ml | 38 ± 0.4 |
| forbesii (1:1 by | | | |
| volume) | | | |
| Magnolia biondii + Notopterygium | 1 | μg/ml | 35 ± 0.3 |
| forbesii (1:1 by | | | |
| volume) | | | |
| Magnolia biondii + Notopterygium | 10 | μg/ml | 32 ± 0.1 |
| forbesii (1:1 by | | | |
| volume) | | | |
| Magnolia biondii + Agastache | 0.1 | μg/ml | 34 ± 0.2 |
| rugosa + Notopterygium forbesii | | | |
| (1:1:1 by volume) | | | |
| Magnolia biondii + Agastache | 1 | μg/ml | 28 ± 0.3 |
| rugosa + Notopterygium forbesii | | | |
| (1:1:1 by volume) | | | |
| Magnolia biondii + Agastache | 10 | μg/ml | 24 ± 0.2 |
| rugosa + Notopterygium forbesii | | | |
| (1:1:1 by volume) | | | |

### Example 2

### Inhibitory Activity of Essential Oils on PAF-Induced Platelet Aggregation

A) Preparation of Platelet Suspension

The anticoagulant “acid-citrate-dextrose” (ACD) was added to the blood taken from a New Zealand rabbit in a ratio of 1:6 by volume. The resulting blood was centrifuged at 190 g for 15 min at room temperature. After centrifugation, the supernatant containing platelet-rich plasma (PRP) was taken and 0.2 M EDTA was added. The resulting supernatant was centrifuged at 1000 g for 1.5 min. After centrifugation, the pellet containing RBC and WBC was removed and then PRP was centrifuged at 1000 g for 11 min. After centrifugation, the supernatant was removed and the platelet pellet was suspended in Ca²⁺⁻-free Tyrode's buffer (containing 1 mM Ca2+). The platelet was added apyrase and cultured for 15 min at 37° C., and then added EDTA (final concentration was 5 mM) and centrifuged at 1000 g for 6 min. After centrifugation, the platelet pellet was resuspended in Ca²⁺⁻Tyrode's buffer (containing 1 mM Ca2+, pH=7.3) and the turbidity of suspension was adjusted to 0.6 (about 2.5×10⁸ platelets/ml). The turbidity was determined with a Platelet Aggregation Chromogenic Kinetic System.

B) Determination of Platelet Aggregation

0.5 ml of platelet suspension was added to silicon-coated cuvette and put in a Platelet Aggregation Chromogenic Kinetic System and bathed for 2 min at 37° C. The essential oils of herbs were added in platelet suspension. Two min. later, PAF was added to the platelet suspension. The platelet aggregation was continually observed for 4 min. The ratio of aggregation was calculated by the following formula:
Ratio of aggregation %=(absorptivity before adding PAF−absorptivity after adding PAF/(absorptivity before adding PAF−absorptivity of Tyrode's buffer)×100%
The ratio of inhibition was calculated by the following formula:
Ratio of inhibition %=100%−(ratio of aggregation of sample/ratio of aggregation of control×100%)

The IC50 values are shown in below Table 2.

TABLE 2

| Samples | IC₅₀(mg/ml) |
|---|---|
| Magnolia biondii | 41 ± 5.5 |
| Agastache rugosa. | 10.5 ± 0.1 |
| Notopterygium forbesii | 19.5 ± 1.5 |
| Magnolia biondii + | 9 ± 1 |
| Agastache rugosa | |
| (1:1 by volume) | |
| Magnolia biondii + | 27 ± 3.4 |
| Notopterygium forbesii | |
| (1:1 by volume) | |
| Magnolia biondii + | 20 ± 2 |
| Agastache rugosa + | |
| Notopterygium forbesii | |
| (1:1:1 by volume) | |
| CV-3988 | 1.42 |

### Example 3

### In Vivo Assay of Essential Oils from Herbs of the Present Invention

Fifty-six male, 8-10 weeks old, Balb/c mice, purchased from the National Laboratory Animal Breeding and Research Center, Taipei, Taiwan, were used in this example. The mice were randomly divided into 8 groups (7 mice/group). Each group was treated by different process listed as follows:
I) PBS group: The mice were sensitized by intraperitioneal injection and challenged by intranasal challenge with PBS.
II) OVA group: The mice were sensitized by intraperitioneal injection and challenged by intranasal challenge with OVA.
III) Essential oil of Magnolia biondii group (MB group): The mice were sensitized by intraperitioneal injection and challenged by intranasal challenge with OVA and then treated with essential oil of Magnolia biondii.
IV) Essential oil of Agastche rugosa group (AR group): The mice were sensitized by intraperitioneal injection and challenged by intranasal challenge with OVA and then treated with essential oil of Agastche rugosa.
V) Essential oil of Notopterygium forbesii group (NF group): The mice were sensitized by intraperitioneal injection and challenged by intranasal challenge with OVA and then treated with essential oil of Notopterygium forbesii.
VI) Essential oil of Magnolia biondii and Agastche rugosa and Notopterygium forbesii group (MAN group): The mice were sensitized by intraperitioneal injection and challenged by intranasal challenge with OVA and then treated with essential oil of Magnolia biondii and Agastche rugosa and Notopterygium forbesii.
VII) Essential oil of Magnolia biondii and Agastche rugosa group (MA group): The mice were sensitized by intraperitioneal injection and challenged by intranasal challenge with OVA and then treated with essential oil of Magnolia biondii and Agastche rugosa.
VIII) Essential oil of Magnolia biondii and Notopterygium forbesii group (MN group): The mice were sensitized by intraperitioneal injection and challenged by intranasal challenge with OVA and then treated with essential oil of Magnolia biondii and Notopterygium forbesii.

The Ovalbumin (OVA, Sigma A-5378) was used as an allergen to sensitize the mice, and Aluminum potassium sulfate (Sigma A-7167) was used as adjuvant. The OVA and Aluminum potassium sulfate were dissolved in a PBS buffer. The resulting solution was particularly administered to each mouse on the 1^{st} day, 5^{th} day, 14^{th} day, and 21^{st} day by intraperitioneal injection.

0.1 m/mouse of the solution was administered.

Twenty-one days after sensitization, the mice were continually challenged with OVA solution (25 mg/ml) daily, dissolved in PBS buffer, for three weeks by intranasal challenge. The dosage of administration was 20 μl/mice/day. The essential oil was vaporized in a closed and sterilized chamber with an electro-thermal hot plate, and then the case was filled with essential oil steam. Ten min. after being challenged, the mice were placed into the case for 30 min.

The clinical behaviors of mice were diagnosed on the 21^{st} day, 28^{th} day, 35^{th} day, and 42^{nd} day after challenge.

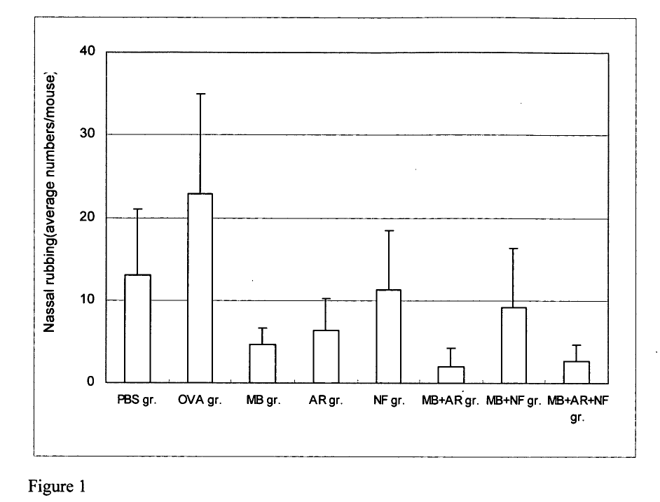

FIG. 1 shows the average number of nasal rubbings counted during the 10 min. after the mice being challenged by intranasal challenge with OVA. The results show that the average number of nasal rubbings significantly decreased after the mice were treated with essential oil of herbal medicine.

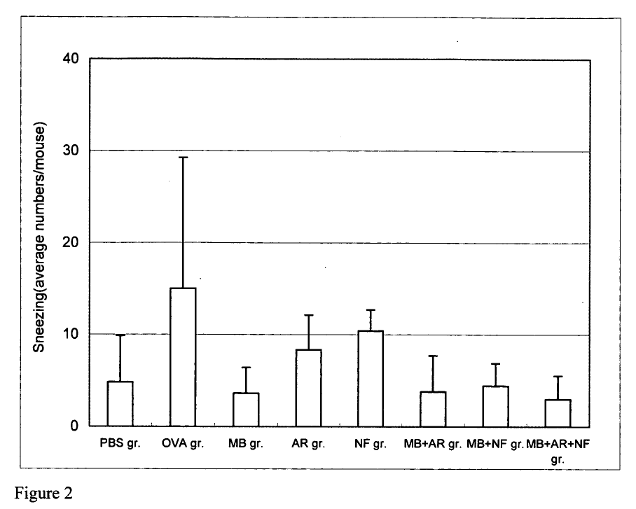

FIG. 2 shows the average number of sneezes counted during the 10 min. after the mice being challenged by intranasal challenge with OVA. The results show that the average numbers of sneezing significantly decreased after the mice were treated with essential oil of herbal medicine.

What is claimed:

1. A composition for use in the treatment and/or prevention of allergic disease, which compruses the essential oil of HOUXIANG HERBA and the essential oil of XINYI HERB, wherein HOUXIANG HERBA compruses a plant selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indicathe and XINYI HERB compruses a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri.

2. The composition according to claim 1, wherein said HOUXIANG HERB is Agastache rugosa.

3. The composition according to claim 1, wherein said XINYI HERB is Magnolia biondii.

4. The composition according to claim 1, wherein said the essential oil of HOUXIANG HERB in the composition ranges from 0.05% to 0.95% by volume, and the essential oil of XINYI HERB in the composition ranges from 0.05% to 0.95% by volume.

5. The composition according to claim 1, wherein said allergic disease includes allergic rhinitis, allergic dermatitis, allergic pneumonia, allergic conjunctivitis, allergic dermatitis, allergic sinusitis, allergic asthma, allergic bronchitis, allergic arthritis, and allergic arteritis and food allergies.

6. The composition according to claim 1, wherein said allergic disease is allergic rhinitis.

7. A composition for use in the treatment and/or prevention of allergic disease, which compruses the essential oil of QIANGHUO HERBA and the essential oil of XINYI HERB, wherein QIANGHUO HERBA compruses a plant selected from the group consisting of Notopterygium forbesii and Notopterygium incisum and XINYI HERB compruses a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri.

8. The composition according to claim 7, wherein said QIANGHUO HERB is Notopterygium forbesii.

9. The composition according to claim 7, wherein said XINYI HERB is Magnolia biondii.

10. The composition according to claim 7, wherein said the essential oil of QIANGHUO HERB in the composition ranges from 0.05% to 0.95% by volume, and the essential oil of XINYI HERB in the composition ranges from 0.05% to 0.95% by volume.

11. The composition according to claim 7, wherein said allergic disease includes allergic rhinitis, allergic dermatitis, allergic pneumonia, allergic conjunctivitis, allergic dermatitis, allergic sinusitis, allergic asthma, allergic bronchitis, allergic arthritis, and allergic arteritis and food allergies.

12. The composition according to claim 7, wherein said allergic disease is allergic rhinitis.

13. A composition for use in the treatment and/or prevention of allergic disease, which comprises the essential oil of HOUXIANG HERBA, the essential oil of QIANGHUO HERB and the essential oil of XINYI HERB, wherein HOUXIANG HERB comprises a plant selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indicathe; QIANGHUO HERBA comprises a plant selected from the group consisting of Notopterygium forbesii and Notopterygium incisum; and XINYI HERB comprises a plant selected from the group consisting of Magnolia biondii, Magnolia denudata Dear., Magnolia denudata Desr., Magnolia pilocarpa, Magnolia liliiflora, Magnolia sargentiana, Magnolia cempbelli Hook., Magnolia martini Dandy, and Magnolia sprengeri.

14. The composition according to claim 13, wherein said HOUXIANG HERB is Agastache rugosa.

15. The composition according to claim 13, wherein said QIANGHUO HERB is Notopterygium forbesii.

16. The composition according to claim 13, wherein said XINYI HERB is Magnolia biondii.

17. The composition according to claim 13, wherein said composition comprises 0.5% to 99.5% by volume of the essential oil of HOUXIANG HERBA, 0.5% to 99.5% by volume of the essential oil of QIANGHUO HERBA, and 0.5% to 99.5% by volume of the essential oil of XINYI HERB.

18. The composition according to claim 13, wherein said allergic disease includes allergic rhinitis, allergic pneumonia, allergic conjunctivitis, allergic dermatitis, allergic sinusitis, allergic asthma, allergic bronchitis, allergic arthritis, and allergic arteritis and food allergies.

19. The composition according to claim 13, wherein said the allergic disease is allergic rhinitis.

20. A method of treating and/or preventing allergic disease, comprising administering an effective and sufficient amount of essential oil of HOUXIANG HERBA to a patient for treating and/or preventing allergic disease, wherein HOUXIANG HERBA comprises the plants selected from the group consisting of Agastache rugosa, Pogostemon cablin, Pogostemon formosanum, Microtoena delavayi, Microtoena insuavis, Microtoena patchouli, Teucrium quadrifarium, Epimeredi indica and Anisomeles indica.

21. The method according to claim 20, wherein said allergic disease includes allergic rhinitis, allergic dermatitis, allergic pneumonia, allergic conjunctivitis, allergic dermatitis, allergic sinusitis, allergic asthma, allergic bronchitis, allergic arthritis, and allergic arteritis and food allergies.

22. The method according to claim 20, wherein said allergic disease is allergic rhinitis.

23. The method according to claim 20, wherein said HOUXIANG HERB is Agastache rugosa.

24. The method according to claim 20, wherein said essential oil is administered to a patient by inhalation delivery, oral delivery, intranasal delivery, transdermal delivery, percutaneous absorption, and/or bath hot.

25. A method of treating and/or preventing allergic disease, comprising administering an effective and sufficient amount of essential oil of QIANGHUO HERBA to a patient for treating and/or preventing allergic disease, wherein QIANGHUO HERBA comprises the plants selected from the group consisting of Notopterygium forbesii and Notopterygium incisum.

26. The method according to claim 25, wherein said QIANGHUO HERB is Notopterygium forbesii.

27. The method according to claim 25, wherein said allergic disease includes allergic rhinitis, allergic dermatitis, allergic pneumonia, allergic conjunctivitis, allergic dermatitis, allergic sinusitis, allergic asthma, allergic bronchitis, allergic arthritis, and allergic arthritis and food allergies.

28. The method according to claim 25, wherein said allergic disease is allergic rhinitis.

29. The method according to claim 25, wherein said essential oil is administered to a patient by inhalation delivery, oral delivery, intranasal delivery, transdermal delivery, percutaneous absorption, and/or bath hot.
